(19) United States
(12) Patent Application Publication
(10) Pub. No.: US 2002/0012650 A1
(43) Pub. Date: Jan. 31, 2002

(54) Hair-treatment compositions

(75) Inventor: Sonja Klein, Hattersheim (DE)

Correspondence Address: CLARIANT CORPORATION, 4331 CHESAPEAKE DR, ATTN: INDUSTRIAL PROPERTY DEPT, CHARLOTTE, NC, 28216, US

(73) Assignee: Clariant GmbH

(21) Appl. No.: 09/803,847

(22) Filed: Mar. 12, 2001

(30) Foreign Application Priority Data
Mar. 11, 2000 (DE) 10012011.3

(51) Int. Cl. A61K 7/075; A61K 7/08

(52) U.S. Cl. 424/70.28

(57) ABSTRACT

The invention provides hair-treatment compositions which comprise at least one quaternary ammonium compound and a pearly luster concentrate comprising a lusterizing component and nonionic, amphoteric and/or zwitterionic surfactants and water. In a preferred embodiment, the pearly luster concentrates comprise titanium dioxide.

### FIELD OF THE INVENTION

The invention relates to hair-treatment compositions, in particular hair cures and hair rinses, comprising quaternary ammonium compounds and having an excellent pearly luster effect.

### BACKGROUND OF THE INVENTION

For the cleansing of the skin and the hair, use is made of surface-active substances which, particularly in the case of daily use, have the disadvantage that they have a strongly defatting action, damage skin and hair and impair the combability of hair.

It is known that cationic surfactants, in particular quaternary ammonium compounds attach to hair damaged by washing, permanent waving and coloring, smooth it and counteract static chargeability during combing.

It is a disadvantage that formulations comprising quaternary ammonium compounds are not brilliant with regard to their appearance, but appear dull and are not very esthetically pleasing.

U.S. Pat. No. 5,019,376 claims cationic oil-in-water haircare emulsions which, upon the addition of (C₁₂-C₁₆)-fatty acids, (C₁₂-C₁₈)-alcohols and a thickener, in particular hydroxyethylcellulose, display pearly luster effects. An unsatisfactory aspect of the compositions is that their storage stability is only mediocre. Pearly luster concentrates produce a reproducible gloss and are therefore more user-friendly.

EP 0 376 083 describes how a mother of pearl-like, silky gloss can be imparted to aqueous compositions by the addition of a pearly luster concentrate comprising lusterizing components, nonionogenic solubility promoters, amphoteric and/or zwitterionic surfactants and low molecular weight polyhydric alcohols.

Hitherto, there have only been aqueous, anionic formulations, such as, for example, shampoos, shower preparations and foam baths, on the market which exhibit a pearly luster effect.

There was therefore the desire for cation-based hair-treatment compositions, in particular hair cures and hair rinses, having a brilliant pearlescence.

### SUMMARY OF THE INVENTION

Surprisingly, it has now been found that hair-treatment compositions comprising at least one quaternary ammonium compound and a pearly luster concentrate comprising a lusterizing component and nonionic, amphoteric and/or zwitterionic surfactants and water, exhibit an excellent pearly luster effect.

### DESCRIPTION OF THE PREFERRED EMBODIMENTS

The invention thus provides hair-treatment compositions comprising at least one quaternary ammonium compound and a pearly luster concentrate comprising a lusterizing component and nonionic, amphoteric and/or zwitterionic surfactants and water.

Suitable ammonium compounds are preferably those compounds which contain one or two (C₈-C₂₀)-alkyl or (C₈-C₂₀)-alkenyl groups. The other groups are (C₁-C₄)-alkyl or (C₂-C₄)-hydroxyalkyl groups. Particularly preferred compounds are cetyltrimethylammonium chloride, PEG-5 stearyl lactate, stearyltrimethylammonium chloride, behenyltrimethylammonium chloride, polydiallyldimethylammonium chloride, distearyldimethylammonium chloride, distearoylethyldimethylammonium chloride, distearoylethylhydroxyethylammonium methosulfate and/or dicocoylethylhydroxyethylammonium methosulfate.

The hair-treatment compositions according to the invention preferably comprise 0.1 to 15% by weight, particularly preferably 1 to 10% by weight, of quaternary ammonium compounds.

The hair-treatment compositions according to the invention preferably comprise 0.5 to 20% by weight, particularly preferably 2 to 7% by weight and especially preferably 3 to 6% by weight of pearly luster concentrate.

The pearly luster concentrates consist primarily of a lusterizing component, one or more surfactants and water.

The pearly luster concentrates preferably comprise 5 to 40% by weight, particularly preferably 20 to 30% by weight, of the lusterizing component and preferably 5 to 55% by weight and particularly preferably 15 to 30% by weight of nonionic, amphoteric and/or zwitterionic surfactants.

In a preferred embodiment, the pearly luster concentrates additionally comprise titanium dioxide, which increases the pearly luster effect.

The pearly luster concentrates preferably comprise 0.01 to 10% by weight, particularly preferably 0.05 to 5% by weight, especially preferably 0.1 to 2% by weight, of titanium dioxide.

Preferred suitable lusterizing components are fatty acid monoalkanolamides, fatty acid dialkanolamides, monoesters or diesters of alkylene glycols, in particular ethylene glycol and/or propylene glycol or oligomers thereof, with higher fatty acids, such as, for example, palmitic acid, stearic acid and behenic acid, monoesters or polyesters of glycerol with carboxylic acids, fatty acids and metal salts thereof, ketosulfones or mixtures of said compounds. Particular preference is given to ethylene glycol distearates and/or polyethylene glycol distearates having on average 3 glycol units.

The pearly luster concentrates used according to the invention can, in addition to the lusterizing component and the surfactants, also comprise up to 10% by weight of polyhydric (C₂-C₈)-alcohols. Preferred polyhydric alcohols are (C₂-C₆)-polyols, in particular ethylene glycol, 1,2- and 1,3-propylene glycol, glycerol, di- and triethylene glycol, erythritol, arabitol, adonitol, xylitol, sorbitol, mannitol and dulcitol.

According to the invention, the pearly luster agents are added to the formulations as pearly luster concentrates. The preparation of such pearly luster concentrates is described in EP 0 376 083.

Surfactants which may be used are nonionogenic, amphoteric and/or zwitterionic interface-active compounds which are notable for a lipophilic, preferably linear alkyl or alkenyl group and at least one hydrophilic group. This hydrophilic group can either be an ionogenic group or a nonionogenic group.

Nonionogenic surfactants contain, as hydrophilic group, e.g. a polyol group, a polyolalkenyl ether group or a combination of polyol and polyglycol ether groups. Preference is given to addition products of from 2 to 30 mol of ethylene oxide, addition products of from 2 to 30 mol of ethylene oxide and up to 5 mol of propylene oxide or addition products of up to 5 mol of propylene oxide with linear fatty alcohols having 8 to 22 carbon atoms, and with alkylphenols having 8 to 15 carbon atoms in the alkyl group, (C₁₂-C₁₉)-fatty acid mono- and diesters of addition products of from 1 to 30 mol of ethylene oxide with glycerol, glycerol mono- and diesters and sorbitan mono- and diesters of saturated and unsaturated (C₈-C₁₈)-fatty acids and ethylene oxide addition products thereof, (C₈-C₁₈)-alkyl mono- and oligoglycosides and ethoxylated analogs thereof, addition products of from 10 to 60 mol of ethylene oxide with castor oil and hydrogenated castor oil, ethoxylated and nonethoxylated mono-, di- and trialkylmonophosphoric esters, in particular mono-, di- and tri(lauryltetraglycol ether)-o-phosphoric esters and mono-, di- and tri(cetyltetraglycol ether)-o-phosphoric esters.

Amphoteric surfactants are understood as meaning those surface-active compounds which, apart from a (C₈-C₁₈)-alkyl or -acyl group in the molecule, contain at least one free amino group and at least one —COOH or —SO₃H group and which are able to form internal salts. Examples of suitable amphoteric surfactants are N-acylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case 8 to 18 carbon atoms in the alkyl group.

Particularly preferred amphoteric surfactants are N-cocoalkylaminopropionate, cocoacylaminoethylaminopropionate and (C₁₂-C₁₈)-alkylsarcosines. Zwitterionic surfactants are understood as meaning those surface-active compounds which carry at least one quaternary ammonium group and at least one —COO— or an SO₃— group in the molecule. Particularly suitable zwitterionic surfactants are betaines, for example, the N-alkyl-N,N-dimethylammonium glycinates, e.g. cocoalkyldimethylammonium glycinates, N-acylaminopropyl-N,N-dimethylammonium glycinates, e.g. cocoacylaminopropyidimethylammonium glycinate, 2-alkyl-3-carboxymethyl-3-hydroxyethylimidazolines having in each case 8 to 18 carbon atoms in the alkyl or acyl group and cocoacylaminoethyl hydroxyethylcarboxymethylglycinate.

The compositions according to the invention can comprise two or more of said surfactants. Preferred surfactant mixtures are mixtures of nonionogenic and zwitterionic or amphoteric surfactants in a weight ratio of from 5:1 to 1:5 or mixtures of nonionogenic surfactants and any mixtures of zwitterionic and amphoteric surfactants in a weight ratio of from 5:1 to 1:5.

The compositions according to the invention can comprise further additives and auxiliaries, such as oily substances, superfatting agents, stabilizers, bodying agents, further thickeners, cationic polymers, silicone compounds, biogenic active ingredients, antidandruff agents, film formers, preservatives, hydrotropic agents, moisture-donating substances, solubilizers, UV absorbers, dyes and fragrances.

Suitable oily substances are all known oils, fats and waxes of mineral, animal, vegetable and synthetic origin. Preferred oil and fat components are diallyl ethers having a total of 12 to 24 carbon atoms, fatty acid esters having a total of 12 to 26 carbon atoms, liquid hydrocarbons having 10 to 32 carbon atoms and mixtures thereof. Suitable fatty acid esters are, for example, methyl palmitate, ethyl oleate, isopropyl myristate, n-hexyl laurate, n-butyl stearate and cetyl/stearyl isononanoate.

Particular preference is given to paraffin oils, vaseline, plant oils, synthetic triglycerides, such as, for example, glyceryl tricaprylate, and also silicone oils.

Superfatting agents which may be used are substances such as, for example, lanolin and lecithin, and also polyethoxylated or acylated lanolin and lecithin derivates, polyol fatty acid esters, monoglycerides and fatty acid alkanolamides. Suitable bodying agents are fatty alcohols having 12 to 22, preferably 12 to 18, carbon atoms, and also partial glycerides.

Further thickeners which may be used are polysaccharides, in particular xantham gum, guar guar, agar agar, alginates, carboxymethylcellulose, hydroxyethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols, such as, for example, pentaerythritol or trimethylolpropane, fatty alcohol ethoxylates or alkyl oligoglucosides, and also electrolytes such as sodium chloride and ammonium chloride.

Suitable silicone compounds are, for example, dimethylpolysiloxane, methyl-phenylpolysiloxanes, cyclic silicones, and also amino-, fatty-acid-, alcohol-, polyether-, epoxy-, fluorine- and/or alkyl-modified silicone compounds, which at room temperature may either be liquid or resin-like.

Biogenic active ingredients are, for example, understood as meaning Bisabolol®, Allantoin®, Phytantriol®, Panthenol®, AHA acids, plant extracts and vitamin complexes.

Antidandruff agents which may be used are Climbazol®, Octopirox®, Oxiconazol® and Zinkpyrethion®.

Customary film formers are chitosan, microcristalline chitosan, quaternized chitosan, polyvinylpyrrolidone, vinylpyrrolidon/vinyl acetate copolymers, polymers of the acrylic acid series, quaternary cellulose derivatives, collagen, hyaluronic acid or salts thereof and similar compounds.

To improve the flow behavior, it is possible to additionally use hydrotropic agents, such as, for example, ethanol, isopropyl alcohol, propylene glycol or glucose.

Suitable preservatives are, for example, phenoxyethanol, formaldehyde solution, parabens, pentanediol or sorbic acid.

Examples of available moisture-donating substances are isopropyl palmitate, glycerol and/or sorbitol.

The total amount of auxiliaries and additives in the compositions is preferably 1 to 50% by weight and particularly preferably 5 to 40% by weight.

The brillance and stability of the pearlescence of the compositions according to the invention is independent of the pH and the viscosity, and also of the use concentrations of the individual components provided they are within the use amounts given above.

The hair-treatment compositions are preferably hair rinses or hair cures.

### EXAMPLES

A number of formulations according to the invention are given below. All percentages are percentages by weight. The examples serve to illustrate the invention in more detail, but not to limit it.

In addition to the components given in the respective examples, the formulations also comprise water and small amounts of perfume oil, preservative and dye solution ad 100% by weight of the formulation.

| Example 1: Cream rinse | |
|---|---|
| Genamin KSL ® | 7% |
| Hostaphat KL 340 D ® | 1.5% |
| Genapol PDB ® | 4% |
| Tylose H 100000YP2 ® | 1.5% |
| Paraffin oil nv | 1% |
| Example 2: Hair cure | |
| Genamin KSL ® | 7% |
| Hostaphat KL 340 D ® | 1.5% |
| Genapol PDB ® | 4% |
| Tylose H 100000YP2 ® | 1.7% |
| Paraffin oil nv | 1% |
| Jojoba oil | 1% |
| Propylene glycol | 0.8% |
| Isopropyl palmitate | 1% |
| Dow Corning 190 ® | 0.8% |
| Extrapon ® | 0.3% |
| Vitamin E | 0.3% |
| Panthenol (vitamin B5) | 0.5% |
| Example 3: Cream rinse | |
| Genamin STAC ® | 3.5% |
| Hostacerin T-3 ® | 1.5% |
| Genapol PDB ® | 4% |
| Tylose H 100000YP2 ® | 1.5% |
| Paraffin oil nv | 1% |
| Example 4: Cream rinse | |
| Genamin CTAC ® | 6% |
| Hostacerin T-3 ® | 1.5% |
| Genapol PDB ® | 3% |
| Tylose H 100000YP2 ® | 1.2% |
| Example 5: Cream rinse | |
| Genamin KSL ® | 7% |
| Hostaphat KL 340 D ® | 1.5% |
| Euperlan PK 3000 ® | 4% |
| Tylose H 100000YP2 ® | 1.5% |
| Paraffin oil nv | 1% |
| Example 6: Hair cure | |
| Genamin KSL ® | 9% |
| Genamin KDM-P ® | 1.5% |
| Hostaphat KL 340 D ® | 1.5% |
| Genapol PDB ® | 4% |
| Tylose H 100000YP2 ® | 1.5% |
| Jojoba oil | 1% |
| Panthenol (vitamin B 5) | 0.5% |

Preparation of the Formulations:

The Preparation is Carried out in the Successive Steps I to VI:

I Swell Tylose in the water at RT with stirring

II Melt oil phase, comprising oil(s), quats, solubility promoters and optionally vitamins at about 75° C.

III Heat water phase (I) to about 75° C.

IV Add water phase (I) to the oil phase (II) and stir until cold

V Add pearly luster concentrate, any die, perfume and plant extract at about 30° C.

VI Adjust the pH

INCI Names of the Commercial Products Used:

| | | |
|---|---|---|
| Genamin KDM-P ® | (Clariant) | behenyltrimethylammonium |
| chloride | | |
| Genamin KSL ® | (Clariant) | PEG-5 stearylammonium |
| | | lactate |
| Genamin CTAC ® | (Clariant) | cetyltrimethylammonium |
| | | chloride |
| Genamin STAC ® | (Clariant) | stearyltrimethylammonium |
| chloride | | |
| Tylose H 100000 YP2 ® | (Clariant) | hydroxyethylcellulose |
| Genapol PDB ® | (Clariant) | glycol distearate, laureth-4, |
| | | cocamidopropylbetaine |
| Hostaphat KL 340 D ® | (Clariant) | trilaureth-4 phosphate |
| Hostacerin T-3 ® | (Clariant) | ceteareth-3 |
| Dow Corning 190 ® | (Dow Corning) | dimethicone copolyol |
| Euperlan PK 3000 ® | (Henkel) | glycol distearate, laureth-4, |
| | | cocamidopropylbetaine |
| Extrapon ® | (Dragoco) | plant extracts |

1. A hair-treatment composition comprising one or more quaternary ammonium compounds and one or more pearly luster concentrates comprising one or more lusterizing components and nonionic, amphoteric and/or zwitterionic surfactants and water.

2. The hair-treatment composition as claimed in claim 1, which comprises 0.1 to 15% by weight of quaternary ammonium compounds.

3. The hair-treatment composition as claimed in claim 1, which comprises 0.5 to 20% by weight of pearly luster concentrate.

4. The hair-treatment composition as claimed in claim 1, wherein the pearly luster concentrates comprise 5 to 40% by weight of the lusterizing component and 5 to 55% by weight of nonionic, amphoteric and/or zwitterionic surfactants.

5. The hair-treatment composition as claimed in claim 1, wherein the pearly luster concentrates comprise titanium dioxide.

6. The hair-treatment composition as claimed in claim 5, wherein the pearly luster concentrates comprise 0.01 to 10% by weight of titanium dioxide.

7. The hair-treatment composition as claimed in claim 1, which comprises, as quaternary ammonium compounds, cetyltrimethylammonium chloride, PEG-5 stearyl lactate, stearyltrimethylammonium chloride, behenyltrimethylammonium chloride, polydiallyidimethylammonium chloride, distearyldimethylammonium chloride, distearoylethyidimethylammonium chloride, distearoyl-ethylhydroxyethylammonium methosulfate and/or dicocoylethylhydroxyethylammonium methosulfate.

8. The hair-treatment composition as claimed in claim 1, which comprises, as lusterizing component, fatty acid monoalkanolamides, fatty acid dialkanolamides, monoesters or diesters of alkylene glycols, fatty acids and metal salts thereof, monoesters or polyesters of glycerol with carboxylic acids and/or ketosulfones.

9. The hair-treatment composition as claimed in claim 8, which comprises, as lusterizing component, ethylene glycol distearates and/or polyethylene glycol distearates having on average 3 glycol units.

10. The hair-treatment composition as claimed in claim 1, which comprises, as nonionic surfactants, addition products of from 2 to 30 mol of ethylene oxide, addition products of from 2 to 30 mol of ethylene oxide and up to 5 mol of propylene oxide or addition products of up to 5 mol of propylene oxide with linear fatty alcohols having 8 to 22 carbon atoms, and with alkylphenols having 8 to 15 carbon atoms in the alkyl group, (C₁₂-C₁₉)-fatty acid mono- and diesters of addition products of from 1 to 30 mol of ethylene oxide with glycerol, glycerol mono- and diesters and sorbitan mono- and diesters of saturated and unsaturated (C₈-C₁₈)-fatty acids and ethylene oxide addition products thereof, (C₈-C₁₈)-alkyl mono- and oligoglycosides and ethoxylated analogs thereof, addition products of from 10 to 60 mol of ethylene oxide with castor oil and hydrogenated castor oil and/or ethoxylated and nonethoxylated mono-, di- and trialkylmonophosphoric esters, as amphoteric surfactants, N-acylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and/or alkylaminoacetic acids having in each case 8 to 18 carbon atoms in the alkyl group, and as zwitterionic surfactants, N-alkyl-N,N-dimethylammonium glycinates, N-acylaminopropyl-N, N-dimethylammonium glycinates, 2-alkyl-3-carboxylmethyl-3-hydroxyethylimidazolines having in each case 8 to 18 carbon atoms in the alkyl or acyl group and/or cocoacylaminoethyl hydroxyethylcarboxymethylglycinate.

11. The hair-treatment composition as claimed in claim 1, which additionally comprises oily substances, superfatting agents, stabilizers, bodying agents, further thickeners, cationic polymers, silicone compounds, biogenic active ingredients, antidandruff agents, film formers, preservatives, hydrotropic agents, moisture-donating substances, solubilizers, UV absorbers, dyes and/or fragrances.

12. The hair-treatment composition as claimed in claim 1, which comprises hydroxyethylcellulose as thickener.

13. The hair-treatment composition as claimed in claim 1, which is a hair cure or a hair rinse.
